(19) United States
(12) Patent Application Publication
(10) Pub. No.: US 2026/0363171 A1
(43) Pub. Date: Nov. 19, 2026

(54) METHOD FOR THE ONLINE ANALYSIS OF THE HARVESTED CROPS

(71) Applicant: CLAAS Selbstfahrende Erntemaschinen GmbH, Harsewinkel (DE)

(72) Inventor: Ralf MARBACH, Versmold (DE)

(21) Appl. No.: 19/677,542

(22) Filed: May 14, 2026

(30) Foreign Application Priority Data
May 14, 2025 (DE) 10 2025 118 541.7

(51) Int. Cl. A01D 41/127; G01N 1/04; G01N 15/14; G01N 15/1434; G01N 33/02

(52) U.S. Cl. CPC A01D 41/1277; G01N 1/04; G01N 15/1434; G01N 33/02; G01N 2015/1493

(57) ABSTRACT

The present invention relates to a method for the online analysis of harvested crops, which is picked up and processed by a self-propelled combine harvester, wherein a crop stream is conveyed by a grain elevator from a conveying and cleaning device (4) to a grain tank of the combine harvester, wherein a sample grain stream from the grain elevator in a bypass configuration, a sample grain stream is extracted from the crop stream passing through the grain elevator, and crop properties of the grains contained in the sample grain stream are determined by a measuring configuration of the sample-taking device with an evaluation unit, comprising the method steps:
separation of individual grains as a sample grain stream from the crop stream using a sampling device,
detection of the grain sizes of the grains in the sample grain stream by an optical sensor configuration downstream of the sampling device and classification into several weight classes, and
determination of constituents of the passing sample grain stream by an optical sensor, wherein the sample grain stream data generated by the optical sensor and corresponding to the detected constituents are transmitted to the evaluation unit in order to determine information regarding the protein shift index (PSI) therefrom, which is evaluated by the evaluation unit to generate at least one recommendation for a subsequent processing step of the crop.

### CROSS-REFERENCE TO RELATED APPLICATION

This application claims priority to German Patent Application No. 10 2025 118 541.7 filed May 14, 2025, which is incorporated by reference in its entirety.

### FIELD OF THE INVENTION

The present invention relates to a method for the online analysis of harvested crops collected and processed by a self-propelled combine harvester, wherein a crop stream is conveyed from a grain elevator via a conveying and cleaning device to a grain tank of the combine harvester, wherein a sample grain stream is extracted from the crop stream passing through the grain elevator using a sampling device arranged in a bypass configuration on the grain elevator, and the properties of the harvested crops of the grains contained in the sample grain stream are determined by a measuring configuration of the sampling device with an evaluation unit. Furthermore, the invention relates to a self-propelled combine harvester.

### BACKGROUND OF THE INVENTION

European Patent 4 179 867 A1 discloses a method for analyzing harvested crops collected by a self-propelled combine harvester. To analyze the composition of the grain, a bypass device is used to divert a partial stream of harvested crops from the main stream of harvested crops-which is conveyed by the grain elevator and consists of grains- and analyze it using a measuring device located within the bypass device. To this end, the measuring device uses a near-infrared sensor that records mass-proportional absorption spectra. Based on this measurement data, the water content, protein content, carbohydrate content, and/or oil content of the partial stream of harvested crops is determined and extrapolated to the total mass in the grain tank.

Based on the prior art described above, the objective of the invention is to further develop a method for the online analysis of the type of harvested crop mentioned at the outset, which is characterized by a detailed analysis of the harvested crop during the ongoing harvesting process.

### SUMMARY OF THE INVENTION

The problem described above is solved by a method for the online analysis of harvested crops collected and processed by a self-propelled combine harvester, wherein a crop stream is conveyed from a grain elevator via a conveying and cleaning device to a grain tank of the combine harvester, wherein a sample grain stream is extracted from the crop stream passing through the grain elevator using a sampling device arranged in a bypass configuration on the grain elevator, and the properties of the harvested crops of the grains contained in the sample grain stream are determined by a measuring configuration of the sampling device with an evaluation unit. Furthermore, the problem is solved by a self-propelled combine harvester for collecting and processing harvested crops, which comprises: a grain elevator for conveying a flow of harvested crops from a conveying and cleaning device to a grain tank; and a sample-taking device arranged on the grain elevator, which is configured to take and determine crop properties of a sample grain stream taken from the crop stream conveyed by the grain elevator, wherein the sample-taking device arranged in a bypass configuration on the grain elevator comprises a measuring configuration with an evaluation unit, wherein the sampling device comprises a rotatably driven sampling device for the continuous separation of individual grains as a sample grain stream from the crop stream, an optical sensor configuration arranged downstream of the sampling device for detecting the size of the separated grains in the sample grain stream, and an optical sensor arranged downstream of the optical sensor configuration, which is designed and configured to determine the constituents of the sample grain stream. Advantageous further embodiments are the subject of dependent claims.

According to one aspect of the disclosure, a method is proposed for the online analysis of harvested crops that is collected and processed by a self-propelled combine harvester, wherein a stream of harvested crops material is conveyed by a grain elevator from a conveying and cleaning device to a grain tank of the combine harvester, wherein a sample grain stream is extracted from the crop stream passing through the grain elevator using a sample extraction device arranged in a bypass configuration on the grain elevator, and crop properties of the grains contained in the sample grain stream are determined by a measuring configuration of the sample extraction device in conjunction with an evaluation unit. The method includes the following steps:
separation of individual grains as a sample grain stream from the crop stream using a sampling device,
detection of the particle sizes of the particles in the sample stream by an optical sensor configuration located downstream of the sampling device, and classification into several weight classes, and
determination of the constituents of the passing sample grain stream by an optical sensor located downstream of the separation device, wherein the sample grain stream data generated by the optical sensor—corresponding to the detected constituents—is transmitted to the evaluation unit to derive information regarding protein shift (PSI), which is evaluated by the evaluation unit to generate at least one recommendation for a subsequent processing step of the harvested crop.

This information on protein displacement indicates the expected percentage of protein in the dry matter of the grain processed by the combine harvester, relative to grain size. In particular, by determining the protein shift data for different grain tank loads, these values can be summed on a mass-weighted basis, allowing for a more accurate and complete determination of the protein shift for the total tonnage.

Preferably, a recommendation for fractionating the harvested crop can be generated as at least one recommendation for the subsequent processing stage. Depending on specific requirements, the combine harvester can perform metrological fractionation during the harvesting process to ensure compliance with protein content specifications for subsequent grain processing, such as flour production or use in the brewing process. In other words, the outcome of so-called “sharp screening”—a mechanical screening process in which grains of a specific weight class are separated based on their high or low protein content relative to their weight—can be predetermined before the harvested crop is sold by the operator of the self-propelled combine harvester or the owner of the harvested grain.

In particular, fractionation can be performed based on the specific proportions of certain components within a given weight class.

According to a further embodiment, the at least one generated recommendation can be transmitted to a remote database, where the at least one recommendation is stored and made available to authorized third parties. The operator of the self-propelled combine harvester or the owner of the harvested grain may, by registering, gain access to the data determined through the method in order to retrieve at least one recommendation for action. This recommendation for action serves as an indicator of the economic benefits of a subsequent screening process designed to modify—that is, to increase or decrease—the protein content by selectively separating a specific weight fraction before the crop is sold.

In order to determine the protein shift with sufficient accuracy, the grains detected by the optical sensor configuration are classified into at least six weight classes. The use of at least six weight classes serves to approximate the expected distribution of particle sizes and the resulting distribution of particle weight relative to the mass flow rate conveyed by the grain elevator.

In particular, individual grains that exceed or fall below a crop-specific threshold value for grain size and/or a crop-specific threshold value for grain weight can be identified and assigned to a common weight class. This makes it suitable for crops with very small seeds, such as those typical of rapeseed, flaxseed, or grasses; for crops characterized by small or medium-sized grains, such as wheat or barley; and for crops characterized by large grains, such as corn or soybeans, a maximum size or a minimum size, or a maximum weight or a minimum weight, may be specified as the basis for the analysis. This enables the sample grain stream to be optically screened based on preferences regarding its components, so that it can be adapted to the requirements of a subsequent processing stage of the harvested crop through preparatory measures. The determination of the fractionation result can thus be integrated into the analytical process.

Furthermore, within a specified measurement period, a predetermined number of consecutive measurements can be performed at a specified measurement interval to determine the particle size, wherein a software algorithm is used by the evaluation unit to generate a histogram of the particle size distribution in the sample particle stream.

In particular, to determine the protein shift, a linear regression equation can be used according to the formula

[(N(t)] _{(m×6)}×m̅_{(6×6))}×P %_{(6×1)}=mP(t)_{(m×1)}

is generated, wherein the matrix N(t) comprises the number of grains in each measurement within the measurement period that are distributed across one of at least six weight classes, the diagonal matrix m̅ contains predetermined values that reflect the average grain weight of each of the at least six weight classes, the vector mP(t) contains values for the protein mass that were measured collectively by the sensor for ingredient analysis within each given measurement interval for all grains contained in the sample grain stream, and the vector P % comprises the protein content concentration to be determined within each of the at least six weight classes, which is solved for the vector P % by the evaluation unit by means of linear regression analysis.

In accordance with a further embodiment, individual grains can be separated from the sample grain stream using a separation device arranged downstream of the optical sensor configuration, based on the evaluation performed by the evaluation unit of signals provided by the optical sensor configuration for the purpose of size discrimination. In this process, the determination of protein shift data by the evaluation unit using linear regression analysis can be preceded by the targeted separation of grains within a specific weight class. The subsequent determination of protein shift information by the evaluation unit is therefore limited to the processing of data from at least five weight classes.

Furthermore, the grains can be separated from the sample grain stream by the separation device based on a threshold value that can be set according to the crop.

Preferably, parameters can be determined using a sensor unit located upstream or downstream of the optical sensor—which comprises one or more sensors employing different sensor technologies—to infer the presence of broken grains, grain health, impurities, grain surface condition, and/or grain color in the sample grain stream.

The problem stated at the outset is further solved by a self-propelled combine harvester having the features described herein.

In another embodiment, a self-propelled combine harvester for collecting and processing harvested crops is proposed, which is constructed and arranged to carry out the method described herein, comprising:
a grain elevator for conveying a flow of harvested crops from a conveying and cleaning device to a grain tank, and
a sample-taking device arranged on the grain elevator, which is configured to take and determine crop properties of a sample grain stream taken from the crop stream conveyed through the grain elevator, wherein the sample-taking device, arranged in a bypass configuration on the grain elevator, comprises a measuring configuration with an evaluation unit, wherein the sampling device comprises a rotatably driven sampling device for the continuous separation of individual grains as a sample grain stream from the crop stream, an optical sensor configuration arranged downstream of the sampling device for detecting the size of the separated grains in the sample grain stream, and an optical sensor arranged downstream of the optical sensor configuration, which is designed and configured to determine the constituents of the sample grain stream. Reference is made to the descriptions of the method for online analysis of harvested crops as disclosed in the invention.

The rotatably driven sampling device for the continuous separation of individual grains as a sample grain stream—which has a substantially constant mass flow of a preset magnitude—from the crop stream ensures that the sample grain stream is formed essentially as a single layer of separated grains or can subsequently be easily formed into a single layer, so that, ideally, an optical analysis of each individual grain in the sample grain stream is enabled by the measuring configuration.

In this context, “separated” means that the optical sensor configuration used to measure the size of the separated grains can, ideally, optically detect each grain in the sample stream.

According to a further embodiment, the extraction device may be designed as at least one rotatably driven cam wheel with at least one circumferential row of cams, or as a rotary vane wheel. Using at least one cam wheel, a defined time interval between the discharge of individual grains can be set, depending on the number of cam rows. In contrast, at a given rotational speed and working width, the rotary feeder allows for a higher flow rate—that is, a higher throughput of individual grains—than the cam pump. Each cell of the rotary feeder holds more grains than a single cam on the cam wheel. The use of at least one cam wheel or cell wheel allows for easy adjustment—by simply replacing it—to accommodate different types of crops that vary in average grain size.

Preferably, at least one cam wheel or rotary feeder is driven at a speed that provides a mass flow rate in the range of 10 g/s to 100 g/s, which is used to feed the sample particle stream to the optical sensor configuration. The preferred mass flow rate for the sample stream is between 20 g/s and 50 g/s. Particularly at mass flow rates in these regions, the two most important grain crops-wheat and barley-exhibit flow characteristics that allow the optical sensor configuration to determine the grain sizes in the passing sample flow with the highest possible precision. At mass flow rates in the preferred range of 20 g/s to 50 g/s, the grains being extracted flow past the optical sensor configuration essentially in a one-dimensional layer.

Preferably, two or more cam wheels may be arranged coaxially side by side, with the at least one circumferential row of cams on each cam wheel being offset in the circumferential direction, or a cam wheel may be provided with two or more rows of cams arranged in a circumferential offset relative to one another. Because the coaxially arranged rows of cams are offset, the cam wheels can pick up the individual grains at staggered intervals and feed them to the downstream optical sensor configuration for size measurement.

In particular, the optical sensor configuration may include at least one through-beam sensor, at least one retro-reflective sensor, and/or at least one photoelectric sensor. The optical sensor configuration may be configured as at least one through-beam sensor and/or at least one diffuse-reflection sensor if the stream of sample grains consists essentially of a single layer of grains that pass through the optical sensor configuration at essentially uniform intervals. In this context, the optical sensor configuration may be designed, for example, as at least one LED through-beam sensor or as at least one LED retro-reflective sensor. In flow conditions where the layer thickness of the sample grain stream is uneven and/or the distances between individual grains in the sample grain stream are uneven and/or too small, it is advisable to design the optical sensor configuration as at least one photoelectric sensor, e.g., a laser line-scanning sensor, in order to be able to measure the size of the separated grains in the sample grain stream even under less than ideal conditions.

If, on the other hand, the grains in the sample stream come into continuous contact and overlap in multiple layers, the optical sensor configuration may be configured as at least one camera with flash illumination. If the optical sensor configuration is implemented as at least one camera with flash illumination, at least one artificial neural network can be used to perform a quantitative analysis of the captured image data.

It is advantageous for the grains dispensed by the dispensing device to be fed into a hollow cylindrical body oriented substantially vertically, which consists at least in part of glass or another transparent material. The grains can flow through the body along its walls and/or move through the hollow cylindrical body in free fall. The optical sensor configuration is positioned adjacent to the hollow cylindrical body so that it can optically detect the flow of sample grains.

Preferably, the evaluation unit can be connected to an external database to which at least one recommendation for a subsequent processing step of the harvested crop can be transmitted; this recommendation is determined by the evaluation unit based on information regarding protein shifts, and the at least one recommendation is stored in the external database in a manner accessible to authorized third parties.

Preferably, the evaluation unit may be designed and configured to evaluate the signals received from the optical sensor configuration in order to identify grains that exceed or fall below a preset threshold value for grain size and/or a preset threshold value for grain weight. The threshold value for grain size and/or the grain weight derived from the grain size may be specified based on the specific crop. This makes it possible to distinguish between crop types characterized by very small grains—such as rapeseed, flaxseed, or grasses—for crops characterized by small or medium-sized grains, such as wheat or barley, and for crops characterized by large grains, such as corn or soybeans, a maximum size or a minimum size, or a maximum weight or a minimum weight, may be specified, which is used to control the separation device. This enables the sample grain stream to be optically screened based on preferences regarding its components, so that it can be adapted to the requirements of a subsequent processing stage of the crop through preparatory measures.

### BRIEF DESCRIPTION OF THE DRAWINGS

The present invention will be explained in more detail below with reference to an exemplary embodiment shown in the drawings. The following is shown in the figures below:

FIG. 1 is a schematic and illustrative side view of a combine harvester;

FIG. 2 is schematic and illustrative representation of a sample collection device;

FIG. 3 is a schematic and illustrative representation of the sample-taking device arranged in a bypass configuration on the grain elevator; and

FIG. 4 is a simplified flowchart illustrating the method steps involved in the online analysis of the harvested crop.

### DETAILED DESCRIPTION

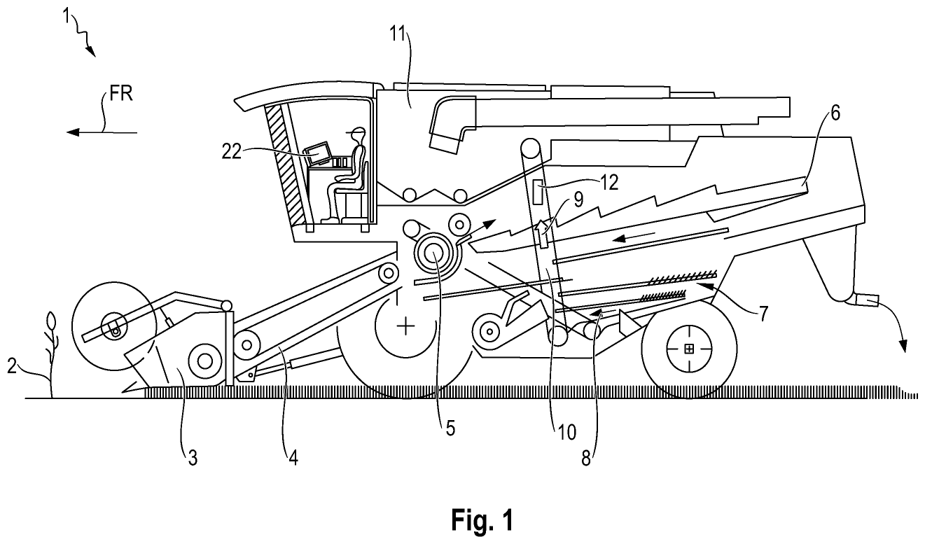

FIG. 1 shows a schematic, illustrative side view of a self-propelled combine harvester 1 designed to collect and process harvested crops 2. During harvesting operations, combine harvester 1 moves forward FR through a crop stand 2, picking up the harvested crop 2 using a crop-specific front attachment 3, such as a cutting unit or a corn header. The harvested crop 2 is conveyed to the threshing unit 5 via an inclined conveyor 4. In the threshing unit 5, a separator 6, and a conveying and cleaning device 7, the grains 8, 8a contained in the harvested crop 2 are separated from the remaining harvested crop 2, resulting in a crop stream 9 consisting of the grains 8, 8a.

The combine harvester 1 is equipped with a grain elevator 10 for conveying the crop stream 9, consisting of grains 8, 8a, from the conveying and cleaning device 7 to a grain tank 11 of the combine harvester 1. A sampling device 12 is arranged in a bypass configuration on grain elevator 10. The sampling device 12 collects a stream of grain samples 13 from the crop stream 9 passing through the grain elevator 10. The sample grain stream 13 flows from the grain elevator 10 into the sample-taking device 12 and passes through it. Upon exiting the sample-taking device 12, the sample grain flow 13 is returned to the grain elevator 10.

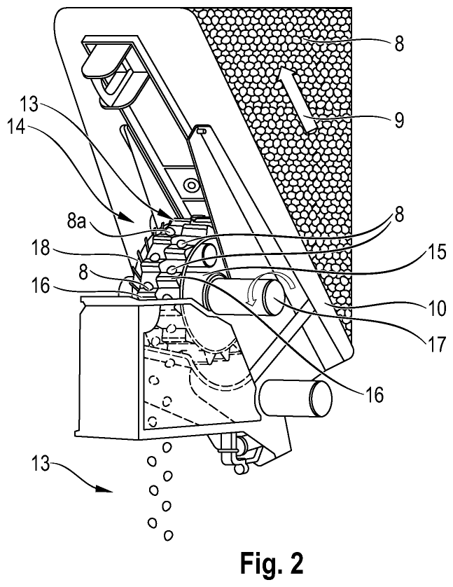

The illustration in FIG. 2 shows, schematically and by way of example, a rotatably driven sampling device 14 of the sample-taking device 12, which is arranged in a bypass configuration on the grain elevator 10. In the illustrated exemplary embodiment, the discharge device 14 is located on the side of the grain elevator 10 facing the direction of travel FR. The grain elevator 10 features a substantially convex bulge with an opening through which the discharge device 14 intermittently engages the crop stream 9 passing through the grain elevator 10. The essentially convex bulge forms a depression in the wall of grain elevator 10, into which the grains 8 are continuously fed from the crop stream 9. Grains 8, 8a are thus continuously fed to the discharge device 14 for removal

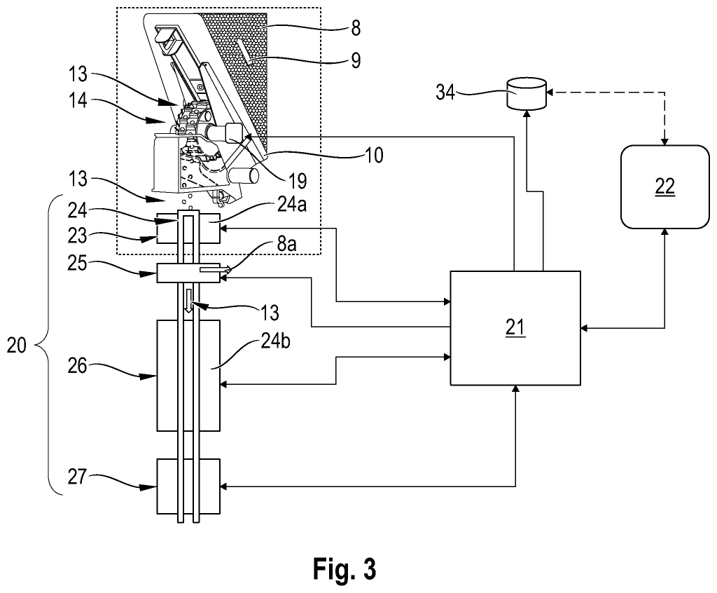

In the embodiment shown in FIG. 2, the removal device 14 is designed as at least one rotatably driven cam wheel 15 with at least one circumferential row of cams 16; in this case, and preferably, it has two rows of cams 15. The cam wheel 15 is mounted on a driven shaft 17 so that it cannot rotate relative to it. To drive shaft 17, a gear 18 may be fixed to shaft 17 such that it meshes with a gear driven by an actuator. Alternatively, shaft 17 can be driven directly by an actuator, as illustrated in FIG. 3.

Preferably, two or more cam wheels 15 may be arranged coaxially side by side on the shaft 17, wherein the at least one circumferential row of cams 16 of each cam wheel 15 is offset in the circumferential direction. Alternatively, a cam wheel 15 may be provided with two or more rows of cams 16 arranged in a circumferentially offset manner relative to one another, as shown in FIG. 2.

The rotatably driven extraction device 14 separates individual grains 8, 8a from the crop stream 9 for the continuous extraction of the sample grain stream 13. The at least one cam wheel 15 or the rotary feeder is preferably driven at a speed to provide a mass flow rate in the range of 10 g/s to 100 g/s, preferably in the range of 20 g/s to 50 g/s, with which the sample particle stream 13 is supplied to the optical sensor configuration 23. At mass flow rates in the preferred range between 20 g/s and 50 g/s, the extracted grains 8, 8a flow past the optical sensor configuration 23 essentially in a one-dimensional layer. A one-dimensional layer refers to the arrangement of grains 8, 8a side by side across a surface, with ideally only a single layer of grains 8, 8a in the vertical direction.

The sample grain stream 13 discharged by the sampling device 14 is fed to an optical sensor configuration 23, which is located downstream of the sampling device 14, for measuring the size of the separated grains 8, 8a in the sample grain stream 13. To this end, it is advantageous for the grains 8, 8a discharged by the dispensing device 14 to be fed into a hollow cylindrical body 24 oriented vertically or substantially vertically, which consists at least in part of glass or another transparent material. The optical sensor configuration 23 is positioned adjacent to the body 24 so that it can optically detect the sample grain flow 13 within the body 24. Preferably, the number of hollow cylindrical bodies 24 to which the grains 8 of the sample grain stream 13 are fed may correspond to the number of cam wheels 15 comprising the discharge device 14. In this configuration, a separate optical sensor configuration 23 can be positioned adjacent to each hollow cylindrical body 24.

FIG. 3 shows a schematic and illustrative representation of the sample-taking device 12 arranged in a bypass configuration on grain elevator 10. The sampling device 12 includes a measuring configuration 20 with an evaluation unit 21. The evaluation unit 21 can be connected to a user interface 22 in the cab of the combine harvester 1. Preferably, the user interface 22 includes an input/output unit. Alternatively or additionally, the evaluation unit 21 may be connected to an external data processing device in order to transmit generated data to it for further use.

The optical sensor configuration 23, located downstream of the extraction device 14 and designed to measure the size of the separated grains 8, 8a in the sample grain stream 13, transmits its signals to the evaluation unit 21 for analysis. The evaluation unit 21 is designed and programmed to distinguish individual grains 8, 8a by size using the signals provided by the optical sensor configuration 23. In this process, the grains 8, 8a detected and optically measured by the optical sensor configuration 23 are classified into different weight classes. Based on the specific size of a grain 8, 8a, an approximation of its weight is made in order to classify the grain 8, 8a into one of the weight classes.

The evaluation unit 21 is designed and programmed to evaluate the signals received from the optical sensor configuration 23 in order to identify individual grains 8a in the sample grain stream 13 that exceed or fall below a threshold value for grain size and/or a threshold value for grain weight—both of which can be preset depending on the crop—and to assign them to a common weight class. The crop-specific threshold value can correspond to a maximum or minimum value for grain size.

The grains 8, 8a of the sample grain stream 13, which are fed from the sampling device 14 to the downstream optical sensor configuration 23, are detected and optically measured by the optical sensor configuration 23. The grains 8 and 8a that have been visually detected and measured—with the exception of grains 8a—are classified into at least six different weight classes by the evaluation unit 21 using an algorithm during the evaluation of the signals. The use of at least six weight classes serves to approximate the expected distribution of grain sizes and the distribution of the resulting grain weight relative to the mass flow rate conveyed by the grain elevator 10.

As previously explained, all detected grains 8a that exceed or fall below the threshold value—which can be set based on the specific crop—are sorted into one of at least six weight classes. The remaining weight classes generally have weight intervals of roughly the same size. This allows one to infer, at least approximately, the weight distribution of the grains 8 in the crop stream 9 conveyed by the grain elevator 10.

The hollow cylindrical body 24 may be divided into several segments 24a, 24b, which are arranged along the measuring configuration 20. The individual segments 24a and 24b are made, at least in part, preferably of borosilicate glass, also known as “technical glass”. Borosilicate glass is durable and cost-effective. In addition, borosilicate glass is transparent in both the visible wavelength range and most of the near-infrared (NIR) wavelength range.

The optical sensor configuration 23 comprises at least one through-beam sensor, e.g., an LED through-beam sensor, and/or at least one reflective sensor, e.g., an LED reflective sensor, and/or at least one photoelectric sensor, e.g., a laser line-scanning sensor. The specific design of the optical sensor configuration 23 depends on the uniformity of the sample grain flow 13 and its layer thickness.

If the sample grain stream 13 consists essentially of a single layer of grains 8, 8a that pass through the optical sensor configuration 23 at essentially uniform intervals, the optical sensor configuration may be implemented, for example, as at least one LED through-beam sensor or as at least one LED reflective sensor. In flow conditions where the layer thickness of the sample particle stream 13 is more uneven and/or the distances between the individual particles 8, 8a in the sample grain stream 13 are uneven and/or too small—i.e., in particular where contact occurs—it is advantageous to design the optical sensor configuration 23 as at least one photoelectric sensor, e.g., a laser line-scanning sensor, in order to be able to perform size measurement of the separated grains 8, 8a in the sample grain stream 13 even under these less-than-ideal conditions.

The sampling device 12 includes a separation device 25 that is optionally arranged downstream of the optical sensor configuration 23. The separation device 25 is designed to separate individual grains 8a from the sample grain stream 13 based on the signals evaluated by the evaluation unit 21 and provided by the optical sensor configuration 23 for the purpose of size classification or weight determination. In this process, the grains 8 contained in the sample grain stream 13 were identified by the optical sensor configuration 23 as grains 8a to be separated from the sample grain stream 13, which exceed or fall below the preset crop-specific threshold value.

In particular, the separation device 25 may be designed and configured for the pneumatic separation of the individual grains 8a from the sample grain stream 13. By separating the grains 8 using the removal device 14 and an optional mechanical separation element located downstream of the removal device 14, the separation device 25 can remove grains 8a that are to be sorted out based on their size from the sample grain stream 13 using targeted bursts of air. Preferably, the sorted and separated grains 8a can be returned to the grain elevator 10.

To separate the grains 8a, the at least two segments 24a, 24b of the body 24 are spaced apart from one another in the area of the separation device 25, so that an open transition zone is formed between them. In the open transition zone, the grains 8a—which are to be separated due to their size—can be separated from the rest of the sample grain stream 13 and discharged outward by means of targeted bursts of air emitted by the separator 25. To control the separation device 25, the evaluation unit 21 is connected to it via signal lines.

The sample-taking device 12 further comprises an optical sensor 27 located downstream of the separator 25, which is designed and configured to determine the composition of the grains 8 remaining in the sample grain stream 13. The optical sensor 27, which is located downstream of the separation device 25, is designed as a NIR sensor. The ingredients in the grains 8 are determined using sensor 27, which is designed as a NIR sensor. The crops to be analyzed are primarily cereals, but oilseeds and legumes are also potential candidates. The components of grain 8 to be determined at least qualitatively include carbohydrates, proteins, moisture (water content), and oils.

In an alternative embodiment of the optional separation device 25, this may be designed as a mechanical screen that separates the grains 8a from the sample grain stream 13 according to the threshold value for a maximum or minimum grain size, so that only those grains 8 are fed to the optical sensor 27 that fall below or exceed this crop-specific threshold value.

An optional sensor unit 26 may be connected upstream or downstream of the optical sensor 27. The optional sensor unit 26 can, for example, be positioned between the separator 25 and the optical sensor 27. The sensor unit 26 comprises one or more sensors based on different sensor technologies, which are configured to determine parameters that allow conclusions to be drawn regarding the presence of broken grains, grain health, impurities, grain surface texture, and/or color of the grain 8 in the sample grain stream 13.

The measurement configuration 20 thus comprises the optical sensor configuration 23, the optional separation device 25, the optional sensor unit 26, and the optical sensor 27, which communicate with the evaluation unit 21 either wirelessly or via a wired connection.

Based on the absolute and/or relative values for the components carbohydrates, proteins, moisture, and oils—which are detected by sensor 27 (designed as an NIR sensor) and determined by the evaluation unit 21—and the dimensions of the grains 8, 8a detected by the optical sensor configuration 23 and evaluated by the evaluation unit 21, conclusions can be drawn about the protein content in relation to grain size.

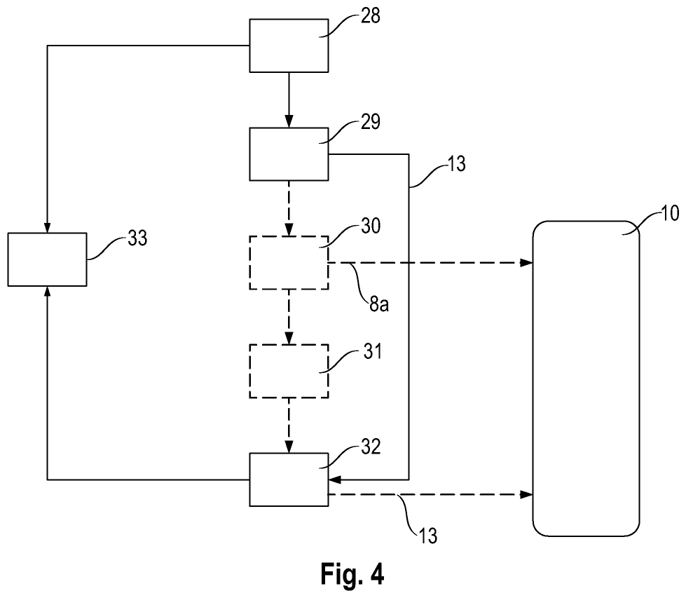

FIG. 4 shows a simplified flowchart illustrating the necessary and optional method steps involved in determining the protein shift index (PSI) during the harvesting process performed by combine harvester 1. It is essential that the sampling device 12 and the method for determining the protein shift information (PSI) be operated while the combine harvester 1 is in operation.

In method step 28, individual grains 8 are separated from the crop stream 9 in the elevator 10 to produce the sample grain stream 13. To this end, the rotatably driven sampling device 14 continuously separates individual grains 8, 8a to form a sample grain stream 13, wherein the sample grain stream 13 has a substantially constant mass flow of a predetermined magnitude.

In method step 29, the size of the grains 8, 8a in the sample grain stream 13 is measured optically using the optical sensor configuration 23 in order to assign the grains 8, 8a to at least six different weight classes. In the case of a camera-based sensor configuration 23, the signals from the optical sensor configuration 23 for optical measurement are evaluated by the evaluation unit 21 using an image processing algorithm. In this process, the number of grains assigned to each weight class (8, 8a) is also determined and stored in a memory unit of the evaluation unit 21. The preferred mass flow rate for the sampled grain stream 13 is preferably between 10 g/s and 100 g/s, and most preferably between 20 g/s and 50 g/s.

Within a measurement period that can be set in the sampling device 12, a predetermined number of consecutive measurements are performed at a configurable measurement interval to determine the grain size. The results of these measurements are assigned to at least six different weight classes, allowing the evaluation unit 21 to generate a histogram of the grain size distribution in the sample grain stream 13 using a software algorithm. The known or adjustable magnitude of the mass flow rate of the extracted sample grain stream 13 results in a known and nearly constant total number of grains 8, 8a per measurement interval, which are optically measured by the optical sensor configuration 23 within the measurement interval.

The method that can be carried out using the sampling device 12 may, as indicated by the dashed lines in FIG. 4, include at least one additional, optional method step 30 and/or method step 31, as explained in more detail below.

First, the remainder of the method is described without the at least one optional method step 30 and/or the optional method step 31.

After being measured optically by the optical sensor configuration 23, the sample grain stream 13 is fed in its entirety—that is, without the grains 8a being separated—to the sensor 27, which is designed as an NIR sensor, for component analysis. Ideally, the NIR sensor detects the composition of all grains 8 and 8a in the sample grain stream 13 that have also passed through the optical sensor configuration 23 within the specified measurement interval.

Ideally, the measurement intervals of the optical sensor configuration 23 and the sensor 27, which is designed as an NIR sensor for ingredient analysis, are precisely synchronized to achieve the highest possible accuracy during the subsequent determination of PSI protein shift information by the evaluation unit 21.

In method step 32, the signals representing the constituents of all grains 8, 8a in the sample grain stream 13 are generated by the sensor 27, which functions as an NIR sensor, and transmitted to the evaluation unit 21 for the determination of the constituents.

As explained above, the specific components include, at a minimum, the protein content, the oil content, and the moisture content, which are determined as relative values based on the dry matter or the total mass of the grains 8, 8a, as detected by the NIR sensor.

After the sample grain stream 13 has passed through the sensor 27 for ingredient analysis, the sample grain stream 13 is returned to the grain elevator 10.

In method step 33, the signals provided by the optical sensor configuration 23 and the sensor 27, which functions as an NIR sensor, are processed by the evaluation unit 21. The evaluation is based on histograms showing the distribution of grain sizes across at least six different weight classes. In addition, the total protein content of sample grain stream 13, based on the dry matter of sample grain stream 13, is known as it passes through sensor 27 for component analysis within the respective measurement interval. Based on this known information, the evaluation unit 21 determines the protein shift (PSI) value, which represents the protein content of the grains 8, 8a within each of the at least six weight classes.

The following is an example of a formula that reflects the relationship described above for at least six weight classes:

[symbol]

(N(t) [symbol] _{(m×6)}×M_{(6×6))}×P %_{(6×1)}=mP(t)_{(m×1)} (1)

$$with$$

$${N ( t )}_{( m \times 6 )} = ⌈ \begin{matrix}{n}_{1 , 1} & {n}_{2 , 1} & {n}_{3 , 1} & {n}_{4 , 1} & {n}_{5 , 1} & {n}_{6 , 1} \\ {n}_{1 , 2} & {n}_{2 , 2} & {n}_{3 , 2} & {n}_{4 , 2} & {n}_{5 , 2} & {n}_{6 , 2} \\ {n}_{1 , 3} & {n}_{2 , 3} & {n}_{3 , 3} & {n}_{4 , 3} & {n}_{5 , 3} & {n}_{6 , 3} \\ ⋮ & ⋮ & ⋮ & ⋮ & ⋮ & ⋮ \\ {n}_{1 , m - 1} & {n}_{2 , m - 1} & {n}_{3 , m - 1} & {n}_{4 , m - 1} & {n}_{5 , m - 1} & {n}_{6 , m - 1} \\ {n}_{1 , m} & {n}_{2 , m} & {n}_{3 , m} & {n}_{4 , m} & {n}_{5 , m} & {n}_{6 , m}\end{matrix} ⌉$$

$${\overline{m}}_{( 6 \times 6 )} = ⌈ \begin{matrix}\overline{m} 1 & 0 & 0 & 0 & 0 & 0 \\ 0 & \overline{m} 2 & 0 & 0 & 0 & 0 \\ 0 & 0 & \overline{m} 3 & 0 & 0 & 0 \\ 0 & 0 & 0 & \overline{m} 4 & 0 & 0 \\ 0 & 0 & 0 & 0 & \overline{m} 5 & 0 \\ 0 & 0 & 0 & 0 & 0 & \overline{m} 6\end{matrix} ⌉$$

$$P {\%}_{( 6 \times 1 )} = ⌈ \begin{matrix}P \%1 \\ P \%2 \\ P \%3 \\ P \%4 \\ P \%5 \\ P \%6\end{matrix} ⌉$$

$$and$$

$${mP ( t )}_{( m \times 1 )} = ⌈ \begin{matrix}mP ( t ) ( t - ( m - 1 ) s ) \\ mP ( t ) ( t - ( m - 2 ) s ) \\ mP ( t ) ( t - ( m - 3 ) s ) \\ ⋮ \\ mP ( t - 1 s ) \\ mP ( t )\end{matrix} ⌉ .$$

The matrix N(t) contains n 8 and 8a grains per measurement, distributed across at least six weight classes. The diagonal matrix m̅ contains predefined values that describe the average grain weight for each of the at least six weight classes. m refers to the number of measurements taken within a measurement period. The vector mP(t) contains the protein masses that were measured collectively by sensor 27 for ingredient analysis within the respective measurement intervals for all grains 8, 8a contained in the sample grain stream 13. The vector P % contains the protein concentration to be determined within each of the at least six weight classes. Knowing the distribution of protein concentration across the respective weight classes makes it possible to determine the result of a subsequent screening of the entire harvested grain 8, 8a contained in at least one filled grain tank 11, which is referred to as protein shift information (PSI). Equation (1) is a linear regression equation that can be solved for the vector P % by evaluation unit 21 using linear regression analysis.

Based on this information regarding the protein shift (PSI), the operator of combine harvester 1 can decide whether to conduct screening at their own expense in order to increase the protein content in the harvested grain 8, 8a, for example, and thereby achieve a higher yield when selling the grain 8, 8a to a grain mill or a middleman. By screening grains 8a, whose grain size exceeds the threshold value and whose protein content relative to grain size is therefore lower than that of the remaining smaller grains 8, the total protein content can be increased in order, for example, to meet the quality criteria for bread wheat. Conversely, in the case of malting barley, for example, the protein content of a batch can be reduced below a maximum level required by the market by sorting out smaller grains 8a, whose weight classes tend to have a higher protein content.

The optional method step 30, which involves the selective separation of individual grains 8a from the sample grain stream 13 using the separation device 25, may be employed in addition to the modification of the method steps 29 and 32 described above.

This is based on the consideration that, during the measurements to be performed by the optical sensor configuration 23 and the sensor 27 for ingredient analysis when detecting grain 8, 8a in the sample grain stream 13, a time discrepancy may occur on a scale that causes the optical sensor configuration 23 to detect different grains 8, 8a than the sensor 27 for ingredient analysis at virtually the same point in time within the measurement interval. This time discrepancy results in a decrease in the accuracy of the determination of the protein shift information (PSI), since the linear regression analysis performed by evaluation unit 21 to solve equation (1) yields only an approximation that is insufficient to provide a sufficiently accurate determination of the protein shift information (PSI) as a basis for action.

The separation device 25 separates the grains 8a whose size exceeds or falls below the specified threshold value for the crop in question before they reach the sensor 27 for ingredient analysis. In this way, the results of both scenarios for a subsequent screening process—that is, the increase in protein content achieved by removing the large grains 8a in the case of wheat, or the reduction in protein content achieved by removing the small grains 8a in the case of malting barley—can be predicted in advance.

By determining the number of grains 8a detected by the optical sensor configuration 23—which, in the case of wheat, for example, exceed the threshold value—and their resulting percentage of the total mass of the sample grain stream 13, the need for the sensor 27 to analyze the composition of this sixth weight class is eliminated.

Equation (1) therefore takes into account only the at least five weight classes of grains 8, the number of which is determined for each weight class, and for which the protein content of the sample grain stream 13—reduced by the removal of grains 8a—is determined by sensor 27 for component analysis. Furthermore, the equation is solved in the same manner by the evaluation unit 21 using linear regression analysis to determine the vector P %. In particular, by using vector P %, information on protein shift index (PSI) can be accurately integrated across multiple grain tank fillings during a harvesting campaign on a single field, so that the PSI of the entire harvested crop can be accurately determined at the end. For this purpose, the vector P %, which is updated every second, for example, can be weighted by the current output signal (tons/hour) from a yield sensor—which is typically installed on combine harvesters and usually measures with a time resolution of approximately 1 second. Both signals can also be averaged over several seconds first (low-pass filtering)—preferably over 10 to 30 seconds—and the weighted integration can then be performed using the averaged data.

If the optional separation device 25 is present, the calculation of the vector P %, i.e., the periodic evaluation of equation (1) can alternatively also be omitted, since the effect of any subsequent screening of the crop can be directly determined from the measurement of the constituents of the optically screened sample grain stream 13—i.e., excluding the separated grains 8a—and can also be approximately integrated.

Method step 31, which is also optional and is carried out by the sensor unit 26, serves, as already explained above, to determine additional parameters of the grain 8, 8a contained in the sample grain stream 13, which allow conclusions to be drawn regarding the presence of broken grain, grain integrity, impurities, surface condition of the grain, and/or color of the grain 8, 8a. This is additional information that can be generated for the operator of combine harvester 1, providing further details on the quality of the crop in grain tank 11 and, consequently, the expected sales revenue.

The at least one recommendation generated by the evaluation unit 21 is transmitted to a remote database 34, in which the at least one recommendation is stored and can be accessed by authorized third parties. The operator of the self-driving combine harvester 1 or the owner of the harvested grain 8, 8a may, by registering, gain access to at least one recommendation for action generated by the method based on the specific information regarding protein shift PSI. In addition to retrieving at least one recommendation for action, it may be provided that the parameters determined by the optional sensor unit 26 regarding the presence of broken grains, grain health, impurities, the surface condition of the grain 8, 8a, and/or the color of the grain 8, 8a can be retrieved from the sample grain stream 13. With regard to section 8a, this applies only to the extent that no separation by the separation device 25 is provided.

### REFERENCE LIST

1 Combine harvester
2 Harvested crop
3 Front attachment
4 Inclined conveyor
5 Threshing mechanism
6 Separation
7 Conveyor and cleaning device
8 Grain
8a Grain
9 Crop stream
10 Grain elevator
11 Grain tank
12 Sampling device

### REFERENCE LIST

13 Sample grain stream
14 Sampling device
15 Cam wheel
16 Row of cams
17 Shaft
18 Gear
19 Actuator
20 Measuring configuration
21 Evaluation unit
22 User interface
23 Sensor configuration
24 Body
25 Separator
26 Sensor unit
27 Sensor
28-33 Method step
34 Database

What is claimed is:

1. A method for the online analysis of harvested crop that is collected and processed by a self-propelled combine harvester, wherein a crop stream is conveyed from a grain elevator via a conveying and cleaning device to a grain tank of the combine harvester, wherein a sample grain stream is extracted from the crop stream passing through the grain elevator using a sampling device arranged in a bypass configuration on the grain elevator, and the properties of the harvested crops of the grains contained in the sample grain stream are determined by a measuring configuration of the sampling device with an evaluation unit, wherein the method comprises:
separation of individual grains as a sample grain stream from the crop stream using a sampling device);
detection of the grain sizes of the grains in the sample grain stream by an optical sensor configuration downstream of the sampling device and classification into several weight classes; and
determination of constituents of the passing sample grain stream by an optical sensor, wherein the sample grain stream data generated by the optical sensor and corresponding to the detected constituents are transmitted to the evaluation unit in order to determine information regarding the protein shift index (PSI) therefrom, which is evaluated by the evaluation unit to generate at least one recommendation for a subsequent processing step of the crop.

2. The method according to claim 1, wherein a recommendation for fractionating the harvested crop is generated as at least one recommendation for the subsequent processing process.

3. The method according to claim 1, wherein the fractionation is carried out based on the determined content fractions of a respective weight class.

4. The method according to claim 1, wherein the at least one generated recommendation is transmitted to a remote database in which the at least one instruction is stored in a manner accessible to authorized third parties.

5. The method according to claim 1, wherein the grains detected by the optical sensor configuration are classified into at least six weight classes.

6. The method according to claim 1, wherein individual grains that exceed or fall below a threshold value for grain size and/or a threshold value for grain weight—which can be set depending on the crop—are identified and assigned to a common weight class.

7. The method according to claim 1, wherein, within a preset measurement period, a predetermined number of consecutive measurements are performed at a preset measurement interval to determine the grain size, wherein the evaluation unit generates a histogram of the particle size distribution in the sample particle stream using a software algorithm.

8. The method according to claim 7, wherein, to determine the protein shift information (PSI), a linear regression equation is established according to the formula, [symbol]
(N(t) [symbol] _{(m×6)}×m̅_{(6×6))}×P %_{(6×1)}=mP(t)_{(m×1)}
wherein the matrix N(t) comprises the number of grains in each measurement within the measurement period that are distributed among at least six weight classes,
the diagonal matrix m̅ contains predetermined values that reflect the average grain weight of each of the at least six weight classes,
the vector mP(t) contains values for the protein mass measured by the sensor for ingredient analysis within each specified measurement interval for all grains contained in the sample grain stream and
the vector P % comprises the concentration of protein content to be determined within each of the at least six weight classes,
which is solved for the vector P % by the evaluation unit by means of linear regression analysis.

9. The method according to claim 1, wherein individual grains are separated from the sample grain stream by means of a separation device arranged downstream of the optical sensor configuration, based on the evaluation performed by the evaluation unit of signals provided by the optical sensor configuration for the size discrimination being performed.

10. The method according to claim 9, wherein the grains (8a) are separated from the sample grain stream by the separating device based on a threshold value that can be set according to the crop.

11. The method according to claim 1, wherein, by means of a sensor unit connected upstream or downstream of the optical sensor, which comprises one or more sensors of different sensor technologies, parameters are determined, based on which conclusions are drawn regarding the presence of broken grains, grain health, impurities, surface condition of the grain, and/or color of the grain in the sample grain stream.

12. A self-propelled combine harvester for collecting and processing harvested crops, which is constructed and equipped to carry out the method according to claim 1, comprising:
a grain elevator for conveying a flow of harvested crops from a conveying and cleaning device to a grain tank); and
a sample-taking device arranged on the grain elevator, which is configured to take and determine crop properties of a sample grain stream taken from the crop stream conveyed by the grain elevator, wherein the sample-taking device arranged in a bypass configuration on the grain elevator comprises a measuring configuration with an evaluation unit, wherein the sampling device comprises a rotatably driven sampling device for the continuous separation of individual grains as a sample grain stream from the crop stream, an optical sensor configuration arranged downstream of the sampling device for detecting the size of the separated grains in the sample grain stream, and an optical sensor arranged downstream of the optical sensor configuration, which is designed and configured to determine the constituents of the sample grain stream.

13. The self-propelled combine harvester according to claim 12, wherein the removal device is designed as at least one rotatably driven cam wheel with at least one circumferential row of cams or as a rotary vane wheel.

14. The self-propelled combine harvester according to claim 12, wherein the optical sensor configuration comprises at least one light barrier, at least one reflective sensor, and/or at least one photoelectric sensor.

15. The self-propelled combine harvester according to claim 12, wherein the evaluation unit can be connected to an external database to which at least one recommended action for a subsequent processing step of the crop can be transmitted, which is determined by the evaluation unit based on information regarding protein shift (PSI), wherein the at least one recommendation is stored in the external database in a manner accessible to authorized third parties.
